(19) United States
(12) Patent Application Publication
(10) Pub. No.: US 2019/0307895 A1
(43) Pub. Date: Oct. 10, 2019

(54) PVP Coated Tellurium Nanorods with Antibacterial and Anticancer Properties

(71) Applicant: Northeastern University, Boston, MA (US)

(72) Inventors: Christopher David Brown, Chelmsford, MA (US); David Medina Cruz, Boston, MA (US); Amit K. Roy, Shrewsbury, MA (US); Thomas J. Webster, Barrington, RI (US)

(21) Appl. No.: 16/375,429

(22) Filed: Apr. 4, 2019

Related U.S. Application Data
(60) Provisional application No. 62/652,890, filed on Apr. 4, 2018.

(51) Int. Cl. A61K 47/69; A61K 9/00; C01B 19/00; A61K 9/14; A61K 9/51; A61K 31/4015

(52) U.S. Cl. CPC A61K 47/6929; A61K 9/0053; C01B 19/002; B82Y 40/00; A61K 9/141; A61K 9/51; A61K 31/4015; A61K 9/0092

(57) ABSTRACT

A nanoparticle formulation of tellurium nanorods. The tellurium nanoparticles are prepared using polyvinylpyrrolidone (PVP), which creates a functionalized coating on the outside of the particles. The nanorods have been shown to have antibacterial properties against both Gram-positive and Gram-negative bacteria, as well as anticancer properties when tested with human melanoma cells.

### RELATED APPLICATION

This application claims the benefit of U.S. Provisional Application No. 62/652,890, filed on Apr. 4, 2018. The entire teachings of the above application are incorporated herein by reference.

### BACKGROUND

With billions of members, the bacterial kingdom is a diverse classification of organisms that contains both beneficial and harmful organisms. While most bacteria are not a cause for concern, there are a substantial number of species that can cause potentially life-threatening infections. Additionally, the global prevalence of bacterial infections has recently received significant attention due to notable increases in antibiotic resistance.

Because of the exponential growth of bacterial populations, mutations often arise that allow bacteria to evolve extremely rapidly. The overuse of antibiotics, partly as a result of inappropriate prescribing and their widespread use in agriculture, has led many types of bacteria to develop antibiotic resistance [1]. As a result, antibiotics such as amoxicillin, methicillin, and vancomycin, have become less effective or not effective at all [2]. Recent data demonstrated that antibiotic-resistant bacteria are more prevalent in medical settings, leading to a rise in healthcare associated infections (HAIs) due to bacteria such as Clostridium difficile and Staphylococcus aureus [3-4]. In 2016, the CDC found that 722,000 HAI cases were reported per year over the past 5 years [5]. Nearly 10% of these patients died as a result of their infections, which can be partially attributed to antibiotic resistant strains of bacteria.

A growing and innovative approach for the treatment of antibiotic resistant bacteria uses nanotechnology. There is an ongoing need to find nanoparticles that are effective against antibiotic-resistant bacteria, and in other antibacterial and anticancer roles.

### SUMMARY

In accordance with an embodiment of the invention, there is provided a nanoparticle formulation of tellurium nanorods. The tellurium nanoparticles are prepared using polyvinylpyrrolidone (PVP), which creates a functionalized coating on the outside of the particles. The nanorods have been shown to have antibacterial properties against both Gram-positive and Gram-negative bacteria, as well as anticancer properties when tested with human melanoma cells.

In one embodiment according to the invention, there is provided a nanoparticle. The nanoparticle comprises tellurium and polyvinylpyrrolidone (PVP), and has a rod shape.

In further, related embodiments, the nanoparticle may have a length of between about 72 nm and about 101 nm, and a width of between about 18 nm and about 26 nm. The nanoparticle may have a length and a width such that an aspect ratio of the length of the nanoparticle to the width of the nanoparticle is between about 2.8 and about 5.3. The nanoparticle may have a zeta potential of between about −25 mV and about −35 mV. The nanoparticle may comprise a surface that is functionalized by the polyvinylpyrrolidone (PVP); and the surface of the nanoparticle may be coated with the polyvinylpyrrolidone (PVP). The nanoparticle may have antibacterial properties against at least one of Gram-positive and Gram-negative bacteria. The nanoparticle may have antibacterial properties against antibiotic resistant bacteria. The nanoparticle may have antibacterial properties against at least one of: Escherichia coli, Staphylococcus aureus, methicillin-resistant Staphylococcus aureus, and multidrug-resistant Escherichia coli. The nanoparticle may have anticancer properties. The nanoparticle may have cytotoxic effects against human melanoma cells.

In another embodiment according to the invention, there is provided a composition. The composition comprises a carrier medium, and a plurality of nanoparticles comprising: (i) tellurium, and (ii) polyvinylpyrrolidone (PVP), the nanoparticles comprising a rod shape. The plurality of nanoparticles is present in the carrier medium in a concentration of between about 10 micrograms per milliliter (μg/mL) and about 100 micrograms per milliliter (μg/mL).

In further, related embodiments, the plurality of nanoparticles may be present in a concentration of between about 25 micrograms per milliliter (μg/mL) and about 100 micrograms per milliliter (μg/mL) in the carrier medium. The carrier medium may comprise a solvent, such as an aqueous solution. The composition may comprise a pharmaceutical composition, and the carrier medium may comprise a pharmaceutically acceptable excipient. The pharmaceutical composition may be suitable for at least one of parenteral administration and oral administration. The nanoparticles of the composition may have a length of between about 72 nm and about 101 nm, and a width of between about 18 nm and about 26 nm. The nanoparticles of the composition may have a length and a width such that an aspect ratio of the length of each nanoparticle to its width is between about 2.8 and about 5.3. The nanoparticles of the composition may have a zeta potential of between about −25 mV and about −35 mV. The nanoparticles of the composition may comprise a surface that is functionalized by the polyvinylpyrrolidone (PVP); and the surfaces of the nanoparticles may be coated with the polyvinylpyrrolidone (PVP). The nanoparticles of the composition may have antibacterial properties against at least one of Gram-positive and Gram-negative bacteria. The nanoparticles of the composition may have antibacterial properties against antibiotic resistant bacteria. The nanoparticles of the composition may have antibacterial properties against at least one of: Escherichia coli, Staphylococcus aureus, methicillin-resistant Staphylococcus aureus, and multidrug-resistant Escherichia coli. The nanoparticles of the composition may have anticancer properties. The nanoparticles of the composition may have cytotoxic effects against human melanoma cells.

In another embodiment according to the invention, there is provided a device comprising an antibacterial or anticancer surface. The device comprises a coating on at least part of a surface of the device, the coating comprising a plurality of nanoparticles comprising: (i) tellurium, and (ii) polyvinylpyrrolidone (PVP), the nanoparticles comprising a rod shape.

In further, related embodiments, the device may comprise medical equipment. The medical equipment may comprise at least one of: a medical implant, a surgical instrument, a medical tubing, and a component of a medical operating theater. The nanoparticles of the coating of the device may have a length of between about 72 nm and about 101 nm, and a width of between about 18 nm and about 26 nm. The nanoparticles of the coating of the device may have a length and a width such that an aspect ratio of the length of the nanoparticle to the width of the nanoparticle is between about 2.8 and about 5.3. The nanoparticles of the coating of the device may have a zeta potential of between about −25 mV and about −35 mV. The nanoparticles of the coating of the device may comprise a surface that is functionalized by the polyvinylpyrrolidone (PVP); and the surfaces of the nanoparticles may be coated with the polyvinylpyrrolidone (PVP).

In another embodiment according to the invention, there is provided a process of preparing a nanoparticle. The process comprises performing a hydrothermal reduction reaction of a tellurium-containing compound with polyvinylpyrrolidone (PVP) and an aqueous solution of an organic compound, thereby producing a solution of nanoparticles comprising: (i) tellurium, and (ii) polyvinylpyrrolidone (PVP), the nanoparticles having a rod shape; and performing a physical purification of the nanoparticles from the solution.

In further, related embodiments, the tellurium-containing compound may comprise a salt of tellurium, such as sodium tellurate. The organic compound may comprise an organic acid, such as ascorbic acid. The physical purification may comprise at least one of: a drying of the solution, a washing, and a lyophilization. The reaction may be performed in a sealed pressure vessel. The nanoparticles produced may have a length of between about 72 nm and about 101 nm, and a width of between about 18 nm and about 26 nm. The nanoparticles produced may have a length and a width such that an aspect ratio of the length of each nanoparticle to its width is between about 2.8 and about 5.3. The nanoparticles produced may have a zeta potential of between about −25 mV and about −35 mV. The nanoparticles produced may comprise a surface that is functionalized by the polyvinylpyrrolidone (PVP); and the surfaces of the nanoparticles produced may be coated with the polyvinylpyrrolidone (PVP).

In another embodiment according to the invention, there is provided a method of treating cancer in a patient in need thereof, comprising administering an effective amount of a pharmaceutical composition comprising a nanoparticle. The pharmaceutical composition comprises (i) a carrier medium; (ii) a plurality of nanoparticles comprising tellurium, and polyvinylpyrrolidone (PVP), the nanoparticles comprising a rod shape; and (iii) a pharmaceutically acceptable excipient. The plurality of nanoparticles is present in the carrier medium in a concentration of between about 10 micrograms per milliliter (μg/mL) and about 100 micrograms per milliliter (μg/mL).

In further, related embodiments of the method of treating cancer in the patient in need thereof, the carrier medium may comprise a solvent. The solvent may comprise an aqueous solution. The method may comprise administering the pharmaceutical composition by parenteral administration. The method may comprise administering the pharmaceutical composition by oral administration. The nanoparticles may have a length of between about 72 nm and about 101 nm, and a width of between about 18 nm and about 26 nm. The nanoparticles may have a length and a width such that an aspect ratio of the length of each nanoparticle to its width is between about 2.8 and about 5.3. The nanoparticles may have a zeta potential of between about −25 mV and about −35 mV. The nanoparticles may comprise a surface that is functionalized by the polyvinylpyrrolidone (PVP). The surfaces of the nanoparticles may be coated with the polyvinylpyrrolidone (PVP). The nanoparticles may have cytotoxic effects against human melanoma cells.

In another embodiment according to the invention, there is provided a method of treating a bacterial infection in a patient in need thereof, comprising administering an effective amount of a pharmaceutical composition comprising a nanoparticle. The pharmaceutical composition comprises: (i) a carrier medium; (ii) a plurality of nanoparticles comprising tellurium, and polyvinylpyrrolidone (PVP), the nanoparticles comprising a rod shape; and (iii) a pharmaceutically acceptable excipient. The plurality of nanoparticles is present in the carrier medium in a concentration of between about 25 micrograms per milliliter (μg/mL) and about 100 micrograms per milliliter (μg/mL).

In further, related embodiments of the method of treating a bacterial infection in a patient in need thereof, the carrier medium may comprise a solvent. The solvent may comprise an aqueous solution. The method may comprise administering the pharmaceutical composition by parenteral administration. The method may comprise administering the pharmaceutical composition by oral administration. The nanoparticles may have a length of between about 72 nm and about 101 nm, and a width of between about 18 nm and about 26 nm. The nanoparticles may have a length and a width such that an aspect ratio of the length of each nanoparticle to its width is between about 2.8 and about 5.3. The nanoparticles may have a zeta potential of between about −25 mV and about −35 mV. The nanoparticles may comprise a surface that is functionalized by the polyvinylpyrrolidone (PVP). The surfaces of the nanoparticles may be coated with the polyvinylpyrrolidone (PVP). The nanoparticles may have antibacterial properties against at least one of Gram-positive and Gram-negative bacteria. The nanoparticles may have antibacterial properties against antibiotic resistant bacteria. The nanoparticles may have antibacterial properties against at least one of: Escherichia coli, Staphylococcus aureus, methicillin-resistant Staphylococcus aureus, and multidrug-resistant Escherichia coli.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1D are Transmission Electron Microscopy (TEM) images of tellurium nanostructures, in accordance with an embodiment of the invention. FIG. 1A shows tellurium nanorods with a low degree of aggregation, found dispersed within the solution. FIG. 1B shows some aggregates that were observed. FIG. 1C shows that the aggregates could be separated with slight sonication. FIG. 1D shows the nanorods at a closer magnification.

FIG. 2 is an Energy Dispersive X-ray Spectroscopy (EDS) of tellurium nanorods, in accordance with an embodiment of the invention.

FIG. 3A is a graph showing growth of a suspension of Escherichia coli over 24 hours in the presence of different concentrations of tellurium nanoparticles. FIG. 3B is a graph showing growth of a suspension of Staphylococcus aureus over 24 hours in the presence of different concentrations of tellurium nanoparticles, in accordance with an embodiment of the invention.

FIGS. 4A through 4C are graphs showing modified Gompertz equation parameters A (in FIG. 4A), μ (in FIG. 4B), and λ (in FIG. 4C) for Escherichia coli and Staphylococcus aureus in the presence of increasing concentrations of PVP-Tellurium nanoparticles, in accordance with an embodiment of the invention.

FIGS. 5A and 5B are graphs showing counting colony assays of Escherichia coli (in FIG. 5A) and Staphylococcus aureus (in FIG. 5B) after being treated for 8 hours with PVP-coated tellurium nanorods, in accordance with an embodiment of the invention.

FIGS. 6A and 6B are graphs showing MTS assays on human dermal fibroblast (in FIG. 6A) and melanoma (in FIG. 6B) cells in the presence of PVP-Tellurium nanoparticles at concentrations ranging from 10-100 μg/mL, in accordance with an embodiment of the invention.

### DETAILED DESCRIPTION

A description of example embodiments follows.

Antibiotic resistance is a predicament that affects more than 2 million people worldwide each year. Through the over-prescription and extensive use of antibiotics, bacteria have generated resistance to many common antibiotic treatments. A promising approach to target antibiotic resistant bacteria is the use of metallic nanoparticles.

Nanoparticles and nanorods have demonstrated significant antibacterial activity, including towards antibiotic resistant bacteria strains [6-8]. These nanoparticles, often composed of silver [9], gold [10], and zinc oxide [11], can be synthesized using a variety of approaches. Traditionally, nanoparticles are synthesized using a series of chemical reactions and physical separations. However, traditional nanoparticle synthesis utilizes harsh chemicals, requires extreme reaction conditions, produces hazardous byproducts, and requires nanoparticle functionalization [12]. Additionally, traditional chemical synthesis often results in aggregates, reducing nanoparticle efficacy in biomedical applications. As a result, “green” synthesis methods have been studied to overcome these issues [13-14].

Nanoparticle synthesis using environmentally safe materials or biological reducing agents can overcome many of the limitations encountered by chemical synthesis. These methods, known as green syntheses, also often utilize organisms, such as bacteria or fungi, to induce rapid generation of metallic nanoparticles [15-17]. Green synthesis of nanoparticles can be performed under less hazardous conditions without producing toxic byproducts. In addition, environmentally safe polymers are often used as coatings and stabilizing agents, which prevents substantial aggregation [18].

A recent material of interest within the nanoparticle community research is elemental tellurium. Tellurium is a metalloid most well-known for its use as an alloy and potent semiconductor [19-20]. Tellurium has no known biological role in humans or other animals [21-22] since it is chemically similar to other chalcogens such as selenium and sulfur. In fact, certain tellurium compounds are mildly toxic to humans and are metabolized by the human body to produce dimethyl telluride as a byproduct [23]. However, tellurium compounds have been studied for their potential anti-inflammatory and anti-cancer effects [24-25]. For example, ammonium trichloro(dioxoethylene-O,O′) tellurate, or AS101, has been shown to slow the growth of cancer cells and to be a potent immunomodulator [26]. Despite this, there has been a lack of other work studying the anti-cancer effects of nanostructured tellurium. Tellurium has been used to produce nanoparticles in many different forms, including nanorods, nanowires, nanoparticles, and quantum dots [27-28]. Many of the approaches that have been used to synthesize these nanoparticles have involved biogenic synthesis methods [29-33]; however, hydrothermal reduction [34] and oxidation [35-36] methods have also been reported. With all of this considered, one potential issue with the use of tellurium nanoparticles for antibacterial purposes is the fact that several strains of bacteria, including Corynebacterium diphtheriae, have been shown to be resistant to the effects of tellurium-containing compounds [37]. Tellurium resistance can also be induced artificially through plasmid-mediated genetic modification of bacteria [38].

To magnify the efficacy of nanoparticles without diminishing their effects, nanoparticle formulations are routinely coated with polymers and other protectants. For example, nanoparticles functionalized with collagen, branched polymers, or targeted peptides all showed increased penetration of negatively-charged bacterial cell walls [39-40]. Another common nanoparticle coating is polyvinylpyrrolidone (PVP), a non-toxic pharmaceutical binder and coating that is commonly used in oral drug delivery. Past research has used PVP to functionalize nanoparticles composed of metals such as silver, copper, and rhodium [41-42]. Metallic nanoparticles have been studied as antibacterial treatments due to their ability to kill antibiotic resistant bacteria. Due to its antibacterial properties, tellurium is a promising material for nanoparticle based bacterial treatment [43].

In experiments in accordance with an embodiment of the invention, an environmentally safe synthesis of tellurium nanoparticles was explored. Rod-shaped tellurium nanoparticles coated with polyvinylpyrrolidone (PVP) were prepared using a facile hydrothermal reduction reaction. Transmission electron microscopy (TEM) images were used to characterize the size and morphology of the nanoparticles and showed a narrow size distribution. In addition, energy dispersive X-ray spectroscopy (EDS) was performed to verify the chemical composition of the nanoparticles.

In the experiments in accordance with an embodiment of the invention, antibacterial assays determined that treatment with nanoparticles at concentrations of 25 to 100 μg/mL induced a decay in the growth of both Gram-negative and Gram-positive bacteria. To determine the effects of the nanoparticles on off-target cells, cytotoxicity assays were performed using human dermal fibroblasts (HDF) and melanoma (skin cancer) cells for durations of 24 and 48 hours. Treatment with nanoparticles at concentrations between 10 and 100 μg/mL showed no significant cytotoxicity towards healthy HDF cells. Contrarily, in melanoma cells, a cytotoxic effect was observed at the same concentrations.

The nanoparticles, in accordance with an embodiment of the invention, therefore possess both anti-cancer and antibacterial effects without being toxic to healthy cells. These properties show that PVP-coated tellurium nanorods can be exploited for the treatment of antibiotic resistant bacterial infections, and in numerous antibacterial and anti-cancer applications.

“Green” synthesis methods of embodiments according to the invention can produce highly functional nanoparticles using less extreme operating conditions than traditional synthesis methods and without producing toxic byproducts. The PVP-coated tellurium nanoparticles produced using the hydrothermal synthesis are rod shaped and exhibit average dimensions of about 80 nm by about 20 nm.

Nanoparticles in accordance with an embodiment of the invention can be used in coatings for medical equipment that is susceptible to bacterial growth. The anti-cancer properties of the nanoparticles allow them to be used in applications in cancer therapeutics. A feature of the nanoparticles is the use of tellurium, an antibacterial, yet under-researched metalloid material. The effects of the nanoparticles on cells, such as HDF and melanoma, allow for a wide range of uses outside of antimicrobial coatings. The nanoparticles can also be used to counter the problem of antibiotic resistance.

The coating of the nanorods is polyvinylpyrrolidone (PVP), an FDA-approved pharmaceutical additive and binder, which is a water-soluble polymer made from the monomer N-vinylpyrrolidone. The nanorods can combine both antibacterial and anticancer properties.

Nanoparticles in accordance with an embodiment of the invention can be used in a variety of different possible applications, including, for example: medical device applications, including antibacterial coatings for medical equipment or tubing; anticancer applications, including potential cancer treatments containing these nanoparticles; antibacterial treatment of hospital acquired infections (HAIs), which represent greater than $28 billion in direct cost to US hospitals and result in 90,000 patient deaths each year; antibacterial coatings for pipes; and cancer therapeutic applications.

A nanoparticle in accordance with an embodiment of the invention comprises tellurium and polyvinylpyrrolidone (PVP), and has a rod shape. The nanoparticle can have a length of between about 72 nm and about 101 nm, and a width of between about 18 nm and about 26 nm. For example, in experiments in accordance with an embodiment of the invention, the nanorods were found to have a width of 22±3.2 nm and length of 86.3±13.8 nm. The nanoparticle can have a length and a width such that an aspect ratio of the length of the nanoparticle to the width of the nanoparticle is between about 2.8 and about 5.3. The nanoparticle may have a zeta potential of between about −25 mV and about −35 mV. For example, in experiments in accordance with an embodiment of the invention, the zeta potential was found to be −30.11±4.52 mV, as synthesized, and, after 60 days, −28.66±3.3 mV, which supports that the nanoparticles are stable after 60 days. The surface of the nanoparticle can be functionalized by the polyvinylpyrrolidone (PVP), for example to have anticancer properties, antibacterial properties, or both. The surface of the nanoparticle can be coated with the polyvinylpyrrolidone (PVP), although the PVP need not necessarily fully coat the nanoparticle. The nanoparticle can have antibacterial properties against Gram-positive bacteria, Gram-negative bacteria, or both; and can have antibacterial properties against antibiotic resistant bacteria. For example, the nanoparticle can have antibacterial properties against one or more of: Escherichia coli, Staphylococcus aureus, methicillin-resistant Staphylococcus aureus, and multidrug-resistant Escherichia coli. For example, in experiments in accordance with an embodiment of the invention, the following strains of bacteria have been used: Escherichia coli (strain K-12 HB101; Bio-Rad, Hercules, Calif.), Staphylococcus aureus (subsp. aureus Rosenbach, ATCC® 12,600™; ATCC, Manassas, Va.), methicillin-resistant Staphylococcus aureus (ATCC 4330; ATCC, Manassas, Va.), and multidrug-resistant Escherichia coli (ATCC BAA-2471; ATCC, Manassas, Va.). The nanoparticle can have anticancer properties, and can, for example, have cytotoxic effects against human melanoma cells. In experiments in accordance with an embodiment of the invention, for example, effects against primary human dermal fibroblasts (ATCC® PCS-201-012™, Manassas, Va.) and melanoma (ATCC® CRL-1619, Manassas, Va.) cells were performed.

A composition according to the invention includes a carrier medium, and the nanoparticles taught herein, with the nanoparticles present in the carrier medium in a concentration of between about 10 μg/mL and about 100 μg/mL, for example in a concentration of between about 25 μg/mL and about 100 μg/mL in the carrier medium. A concentration of between about 10 μg/mL and about 100 μg/mL can, for example, be used for anticancer effects, and the concentration of between about 25 μg/mL and about 100 μg/mL can, for example, be used for antibacterial effects. In experiments in accordance with an embodiment of the invention, bacterial analysis has been performed at 25, 50, 75 and 100 μg/mL, for example, while cell analysis has been performed at 10, 15, 50 and 100 μg/mL, for example.

The carrier medium can be a solvent, such as an aqueous solution. The composition can be a pharmaceutical composition, and can include a pharmaceutically acceptable excipient. The pharmaceutical composition can be suitable for one or more of parenteral administration and oral administration.

A device in accordance with an embodiment of the invention includes an antibacterial or anticancer surface, with a coating on at least part of the surface of the device, the coating including the nanoparticles taught herein. The device can, for example, be medical equipment, such as one or more of: a medical implant, a surgical instrument, a medical tubing, and a component of a medical operating theater.

A process of preparing a nanoparticle in accordance with an embodiment of the invention, includes performing a hydrothermal reduction reaction of a tellurium-containing compound with polyvinylpyrrolidone (PVP) and an aqueous solution of an organic compound, thereby producing a solution of the nanoparticles taught herein. A physical purification of the nanoparticles from the solution is performed. The tellurium-containing compound can be a salt of tellurium, such as sodium tellurate. The organic compound can be an organic acid, such as ascorbic acid. The physical purification can, for example, be a drying of the solution, a washing, and a lyophilization; and can involve no chemical changes to the solution. The reaction can be performed in a sealed pressure vessel.

A method of treating cancer in a patient in need thereof, in accordance with an embodiment of the invention, comprises administering an effective amount of a pharmaceutical composition comprising a nanoparticle taught herein. The pharmaceutical composition comprises (i) a carrier medium; (ii) a plurality of nanoparticles comprising tellurium, and polyvinylpyrrolidone (PVP), the nanoparticles comprising a rod shape; and (iii) a pharmaceutically acceptable excipient. The plurality of nanoparticles is present in the carrier medium in a concentration of between about 10 micrograms per milliliter (μg/mL) and about 100 micrograms per milliliter (μg/mL). The nanoparticles can, for example, have cytotoxic effects against human melanoma cells, or another cancer.

A method of treating a bacterial infection in a patient in need thereof, in accordance with an embodiment of the invention, comprises administering an effective amount of a pharmaceutical composition comprising a nanoparticle taught herein. The pharmaceutical composition comprises: (i) a carrier medium; (ii) a plurality of nanoparticles comprising tellurium, and polyvinylpyrrolidone (PVP), the nanoparticles comprising a rod shape; and (iii) a pharmaceutically acceptable excipient. The plurality of nanoparticles is present in the carrier medium in a concentration of between about 25 micrograms per milliliter (μg/mL) and about 100 micrograms per milliliter (μg/mL). The nanoparticles can, for example, have antibacterial properties against at least one of Gram-positive and Gram-negative bacteria. The nanoparticles can, for example, have antibacterial properties against antibiotic resistant bacteria. The nanoparticles can, for example, have antibacterial properties against at least one of: Escherichia coli, Staphylococcus aureus, methicillin-resistant Staphylococcus aureus, and multidrug-resistant Escherichia coli.

The invention also provides a method of treating a cancer, comprising administering to a subject in need thereof the pharmaceutical composition comprising the nanoparticles of the invention for a time sufficient to treat the cancer.

In addition, the invention also provides a method of treating a bacterial infection, comprising administering to a subject in need thereof the pharmaceutical composition comprising the nanoparticles of the invention for a time sufficient to treat the bacterial infection.

The pharmaceutical compositions of the invention may be administered in combination with one or more additional therapeutic agents, or combinations thereof.

For example, an additional therapeutic agent may be cyclophosphamide, busulfan, dibromomannitol, streptozotocin, dacarbazine (DTIC), procarbazine, melphalan, mechlorethamine, thioepa, chlorambucil, carmustine (BSNU), lomustine (CCNU), mitomycin C, cisplatin and other platinum derivatives, such as carboplatin, thalidomide or a thalidomide analog, lenalidomide or CC4047, a proteasome inhibitor, such as bortezomib or vinca alkaloid, such as vincristine or an anthracycline, such as doxombicin; a proteasome inhibitor, for example, bortezomib, carfilzomib or ixazomib.

As used herein, the “effective amount” of a pharmaceutical composition comprising a nanoparticle refers to the amount needed to perform its intended function, i.e., elicit a desired biological response. As understood by those of ordinary skill in this art, the effective amount of nanoparticle may vary depending on such factors including, the type and severity of the disease, the patient's gender, age, weight and health, the route of administration, etc. The nanoparticles disclosed herein may be formulated in dosage unit form. For example, dosages of the nanoparticles may be from about 0.01 mg/kg/day to about 500 mg/kg/day, for example, from about 0.1 mg/kg/day to about 100 mg/kg/day.

A therapeutically effective amount of the nanoparticles can be administered in a single dose or multiple doses. Further, the dosages of the nanoparticles can be proportionally increased or decreased as needed.

The methods may include administration of a nanoparticle composition, wherein the composition is administered over a period of one week, two weeks, three weeks, a month, two months or longer. For example, disclosed herein are methods of treating cancers or bacterial infections that include administering a nanoparticle composition over a period of at least two weeks, three weeks, one month or administered over a period of about 2 weeks to about 6 months or more, at once, twice, or three times a day, once a week, once every two weeks, once every three weeks, or once every month, and wherein the dose of the active agent at each administration is from about 0.01 mg/kg to about 500 mg/kg, for example, from about 0.1 mg/kg to about 100 mg/kg.

In some embodiments, the additional therapeutic agent is a chemotherapeutic agent. In some embodiments, the other chemotherapeutic agent is capecitabine, oxaliplatin, gemcitabine, 5FU, mitomycin, cisplatin, gemcitabine or a combination of other chemotherapeutic agents.

In some embodiments, the nanoparticles and the other therapeutic agent(s) agent can be administered simultaneously, either in the same composition or in separate compositions, or administered sequentially, i.e., the nanoparticle composition can be administered either prior to or after the administration of the other chemotherapeutic or antibacterial agent. In some embodiments, the administration of the nanoparticle composition and the additional therapeutic agent can be concurrent, i.e., the administration period of the nanoparticle composition and that of the chemotherapeutic or antibacterial agent are simultaneous or overlap with each other. In some embodiments, the administration of the nanoparticle composition and the additional agent are non-concurrent. For example, in some embodiments, the administration of the nanoparticle is terminated before the additional therapeutic agent is administered. In some embodiments, the administration of the other additional therapeutic agent is terminated before the nanoparticle is administered.

In some embodiments, nanoparticles may be administered orally, parenterally, topically (as by powders, creams, ointments, or drops), by injection (e.g., intravenous, subcutaneous or intramuscular, intraperitoneal injection), rectally, vaginally, or by inhalation (as by sprays). In one embodiment, the nanoparticles of the present invention are administered to a subject in need thereof systemically, e.g., by IV infusion or injection.

### Definitions

As used herein, a “nanoparticle” comprises a particle of less than about 1 micron in its largest dimension.

As used herein, a “rod” shape is a three-dimensional solid shape that is longer than it is wide, and can, for example, be a cylindrical or substantially cylindrical shape.

As used herein, a “nanorod” is a nanoparticle that is rod shaped.

As used herein, an “antibacterial” property is the property of inhibiting or killing bacteria.

As used herein, an “anticancer” property is the property of destroying or inhibiting cancer cells.

As used herein an “implant” is a medical device that replaces, supports or enhances a biological structure, and can, for example include a sensory or neurological implant, a cardiovascular implant, an orthopedic implant, an electric implant, a contraception implant, a cosmetic implant, a gastrointestinal implant, a respiratory implant or a urological implant.

As used herein, a “pharmaceutically acceptable excipient” is a substance formulated alongside the active ingredient of a pharmaceutical composition, for long-term stabilization, bulking up, or conferring therapeutic enhancement; and that is shown to be pharmaceutically safe. For example, excipients can include, for example, buffers, acids, bases, salts, solubilizers, preservatives, chelating agents, sugars, amino acids, proteins and solvents. One, several, or all of these can be present in a pharmaceutical composition.

A list of possible buffering agents includes but is not limited to ACES, acetate, ADA, ammonium hydroxide, AMP (2-amino-2-methyl-1-propanol), AMPD (2-amino-2-methyl-1,3-propanediol), AMPSO, BES, BICINE, bis-tris, BIS-TRIS propane, borate, CABS, cacodylate, CAPS, CAPSO, carbonate, CHES, citrate, DIPSO, EPPS, HEPPS, ethanolamine, formate, glycine, glycylglycine, HEPBS, HEPES, HEPPSO, histidine, hydrazine, imidazole, malate, maleate, MES, methylamine, MOBS, MOPS, MOPSO, phosphate, piperazine, piperidine, PIPES, POPSO, propionate, pyridine, pyrophosphate, succinate, TABS, TAPS, TAPSO, taurine (AES), TES, tricine, triethanolamine, Trizma.

Salts can, for example, include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts. Alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine are also included.

Preservatives are often added to biopharmaceutical products to lengthen the shelf live. A representative list includes ascorbic acid, benzoic acid, benzyl alcohol, benzylalkonium chloride, erythorbic acid, propionic acid, sorbic acid, thiodipropionic acid, ascorbyl palmitate, butylated hydroxyanisole, butylated hydroxytoluene, calcium ascorbate, calcium propionate, calcium sorbate, dilauryl thiodipropionate, gum guaiac, methylparaben, metabisulfite, m-cresol, paraben, potassium bisulfite, potassium metabisulfite, potassium sorbate, propyl gallate, propylparaben, sodium ascorbate, sodium benzoate, sodium bisulfite, sodium metabisulfite, sodium propionate, sodium sorbate, sodium sulfite, stannous chloride, sulfur dioxide, tocopherols.

In order to dissolve and maintain dissolution of the pharmaceutical ingredient, solubilizers can be added to the biopharmaceutical composition; PEG, Tween, CMC, and SDS are all possibilities for agents used as solubilizers.

Chelating agents include, but are not limited to, citric acid, tartaric acid, calcium acetate, calcium chloride, calcium citrate, calcium diacetate, calcium gluconate, calcium hexametaphosphate, monobasic calcium phosphate, calcium phytate, dipotassium phosphate, disodium phosphate, isopropyl citrate, malic acid, monoisopropyl citrate, potassium citrate, sodium citrate, sodium diacetate, sodium gluconate, sodium hexametaphosphate, sodium metaphosphate, sodium phosphate, sodium pyrophosphate, tetra sodium pyrophosphate, sodium tartrate, sodium potassium tartrate, sodium thiosulfate, sodium tripolyphosphate, stearyl citrate, and tetrasodium ethylenediamine tetraacetate.

Acid and bases are often added to biopharmaceutical products to adjust pH or to enhance efficacy. Representative acids includes nitric acid, hydrochloric acid, sulfuric acid, perchloric acid, hydrobromic acid, hydroiodic acid, acetic acid, ascorbic acid, boric acid, butanoic acid, carbonic acid, citric acid, formic acid heptanoic acid, hexanoic acid, hydrocyanic acid, hydrofluoric acid, lactic acid, nitrous acid, octanoic acid, oxalic acid, pentanoic acid, phosphoric acid, propanoic acid, sulfurous acid, and uric acid. Representative bases include lithium hydroxide, sodium hydroxide, potassium hydroxide, magnesium hydroxide, calcium hydroxide, alanine, ammonia, dimethylamine, ethylamine, glycine, hydrazine, methylamine, and trimethylamine.

Sugars, including glucose (dextrose), fructose, galactose, ribose, sucrose, lactose, maltose, trehalose, cellobiose, arabitol, erythritol, glycerol, isomalt, lactitol, maltitol, mannitol, sorbitol, and xylitol.

Proteins such as albumin, and peptides can also be included as excipients.

An example of amino acid used as an excipient is L-histidine.

An example of protein used as an excipient is bovine serum albumin (BSA).

As used herein, an “organic acid” is an organic compound with acidic properties, and can include, for example, carboxylic acids, sulfonic acids, alcohols, thiols, enols, and phenols; such as, lactic acid, acetic acid, formic acid, citric acid, oxalic acid, uric acid, and ascorbic acid, such as 1-ascorbic acid.

As used herein, a “zeta potential” is the electrokinetic potential in a colloidal dispersion, which is the electric potential in the interfacial double layer at the location of the slipping plane relative to a point in the bulk fluid away from the interface. For example, the zeta potential of a nanoparticle in accordance with an embodiment of the invention is the potential difference between the dispersion medium, in which the nanoparticle is dispersed, and the stationary layer of fluid attached to the dispersed nanoparticle.

### Examples

There will now be described a set of example experiments, conducted in accordance with an embodiment of the invention.

### 1. MATERIALS AND METHODS

1.1. Tellurium Nanorod Synthesis and Purification

Tellurium nanorods coated with polyvinylpyrrolidone (PVP) were synthesized using a hydrothermal reduction method, reducing sodium tellurate (Na₂TeO₄) to elemental tellurium (Te⁰). Briefly, 40 mL of 20 g/L PVP (Sigma Aldrich, St. Louis, Mo.) in deionized water was mixed with 1.0 gram of 1-ascorbic acid (Sigma Aldrich) and enough sodium tellurate to reach a final concentration of 0.5 mM (Alfa Aesar, Haverhill, Mass.). This solution was stirred for 10 minutes at room temperature, upon which time a color change from clear to yellow-orange was observed. The solution was then transferred into a sealed reaction vessel and placed into a Thermo Scientific Heratherm™ General Protocol Oven at 90° C. for 20 hours.

$${Na}_{2} {TeO}_{4} + {C}_{6} {H}_{8} {O}_{6} + PVP {\to }^{90 ° C . / 20 h} TeNPs - PVP \qquad ( 1 )$$

Equation (1) gives the chemical reaction for the synthesis of tellurium nanoparticles.

The solution was then removed from the oven and allowed to cool naturally to room temperature. The nanorod-containing solution was then transferred to a 50 mL conical centrifuge tube and centrifuged at 11,000 RPM for 20 minutes in an Eppendorf™ Model 5804-R Centrifuge. The supernatant was subsequently removed and discarded. The resulting precipitate was then washed twice with deionized water and once with 200-proof ethanol (Sigma) via centrifugation at 11,000 RPM for 20 minutes. The final nanoparticle pellet was reconstituted in 5 mL of deionized water and placed into a 20 mL glass vial. This vial was frozen at −80° C. for 4 hours before being lyophilized at −80° C. for 24 hours in a FreeZone Plus 2.5 Liter Cascade Console Freeze Dry System. The resulting nanoparticle solids were then weighed and resuspended in deionized water at a stock concentration of 20 mg/mL.

1.2. Instruments and Characterization

Characterization of PVP-coated tellurium nanorods was accomplished using TEM imaging, EDS analysis, and DLS. Initially, transmission electron microscopy was performed. Images were captured via a JEM-1010 TEM (JEOL USA Inc., Peabody, Mass.). For sample preparation, purified nanoparticles were air-dried on 300-mesh copper-coated carbon grids (Electron Microscopy Sciences, Hatfield, Pa.). The sample was then imaged up to a 75,000× magnification with an accelerating voltage of 60.0 kV.

Energy dispersive X-ray spectroscopy analysis was performed using a dedicated EDS detector coupled with a Hitachi S-4800 SEM. Nanoparticle samples were affixed to 300-mesh copper coated carbon grids and placed into an aluminum pin mount. An accelerating voltage of 10.0 kV was used to obtain an elemental spectrum for the nanoparticles.

Lastly, dynamic light scattering (DLS) sizing was performed. The purified nanorod solution was first diluted by a factor of 100 in phosphate buffered saline (PBS). This resulting solution was then sonicated briefly before being transferred to a 1.0 mL quartz cuvette and analyzed. Measurements showed a polydispersity of 0.249, indicating a relatively monodisperse distribution.

1.3. Bacterial Cultures

Strains of both gram negative and gram positive bacteria were used in this study to determine the antibacterial activity of PVP-coated tellurium nanorods. Escherichia coli (strain K-12 HB101; Bio-Rad, Hercules, Calif.) and Staphylococcus aureus (subsp. aureus Rosenbach, ATCC® 12600™; ATCC, Manassas, Va.) bacteria were used. Prior to inoculation, the bacterial cultures were maintained on agar plates at 4° C. Bacteria were introduced into 6 mL of sterile Luria-Bertani (LB) (bioPLUS, bioWORLD) medium in a 15 mL Falcon centrifuge tube and incubated at 37° C./200 rpm for 24 hours. The optical density (OD) of the bacterial cultures was measured at 600 nanometers (nm) using a spectrophotometer (SpectraMax M3, Molecular Devices, Sunnyvale, Calif.). The bacterial suspension was then diluted to a concentration of 10⁶ colony forming units per milliliter (CFU mL⁻¹) and stored at 4° C. until use.

1.4. Antibacterial Effects of Tellurium Nanoparticles

For the antibacterial assay, solutions of tellurium nanorods in autoclaved and sterilized water were prepared in LB medium and combined with an equal volume of either Escherichia coli or Staphylococcus aureus bacterial suspension in a 96-well, clear-bottom plate. The final nanoparticle concentrations of these solutions was between 25 and 100 μg/mL. For untreated controls, 100 μL of each bacteria was mixed with 100 μL of LB medium without nanoparticles and added to the plate. Additionally, a control containing nanoparticles and medium alone was used to observe the innate absorbance of the nanoparticles. The final volume per well was 200 μL. Once the plate was prepared, the absorbance of all samples was measured at 600 nm every 2 minutes for 24 hours.

The resulting bacterial growth curves were shifted to the origin by subtracting the initial value from the entire curve and fitted with the modified Gompertz model. The original Gompertz equation, which describes a sigmoidal growth curve, contains mathematical parameters (a, b, and c) as seen in Equation (2), as opposed to parameters with biological meanings (A, μ, and λ) as seen in Equation (3). Thus, to fit the Gompertz model to the assay data, a parameterization of the growth model was needed. This was done by deriving expressions for the biological parameters as functions of the original mathematical parameters and then substituting them into the original formula. The Gompertz equation, written in terms of mathematical parameters, as,

y=a·exp[−exp(b−ct)] (2)

was consequently modified through a series of derivations to obtain the modified Gompertz equation that was used to describe the bacterial growth curves in terms of biological parameters:

$$y = A \cdot exp ( - exp [ \frac{μ \cdot e}{A} ( λ - t ) + 1 ] ) \qquad ( 3 )$$

The dependent variable y represents the amount of bacteria (as measured by the absorbance reading), A is the maximum possible value of y, μ is the maximal growth rate and λ is the lag time. The parameters A, μ and λ were then estimated according to a least-squares estimation algorithm using a GRG nonlinear solver.

Additionally, colony counting assays were performed to determine the antibacterial effects of tellurium nanorods. Briefly, solutions of tellurium nanorods were prepared in LB medium and added to an equal volume of either Escherichia coli or Staphylococcus aureus bacteria in a 96-well, clear-bottom plate to final nanoparticle concentrations of 25 to 100 μg/mL. The 96-well plate was then placed in a 37° C. incubator for 8 hours. Following incubation, the plate was removed from the incubator and all samples were diluted in sterile PBS to obtain serial dilutions of 100×, 1000×, and 10,000×. Then, 10 μL of each sample was placed onto a labeled agar plate and incubated for 8 hours at 37° C. The total number of colonies for each sample was counted post-incubation.

1.5. In Vitro Cytotoxicity Assay with Tellurium Nanoparticles

Cytotoxicity assays were performed with primary human dermal fibroblasts (ATCC® PCS201-012™, Manassas, Va.) and melanoma (ATCC® CRL-1619, Manassas, Va.) cells. The cells were both grown separately in Dulbecco's Modified Eagle Medium (DMEM; Thermo Fisher Scientific, Waltham, Mass.) supplemented with 10% fetal bovine serum (FBS; ATCC® 30-2020™, Manassas, Va.) and 1% penicillin/streptomycin (Thermo Fisher Scientific, Waltham, Mass.). Cell viability (MTS) assays (CellTiter 96® AQueous One Solution Cell Proliferation Assay, Promega, Madison, Wis.) were carried out to assess cytotoxicity. Cells were seeded onto tissue-culture treated 96-well plates (Thermo Fisher Scientific, Waltham, Mass.) at a concentration of 5000 cells per well in 100 μL of medium. After an incubation period of 24 hours at 37° C. in a humidified incubator with a 5% carbon dioxide (CO₂) atmosphere, the culture medium was aspirated from the wells and replaced with 100 μL of fresh medium containing a defined concentration of tellurium nanoparticles. Experimental controls containing medium alone and HDF cells with medium were also prepared. The plate was then incubated for 24 hours under the same environmental conditions. The culture medium was removed and replaced with 100 μL of MTS solution containing a ratio of 1 part MTS to 5 parts medium. After adding the MTS solution, the 96-well plate was incubated for 4 hours at 37° C. to allow for the reduction of MTS to formazan by viable cells. The absorbance was then measured at 490 nm on an absorbance plate reader (SpectraMax Paradigm Multi-Mode Detection Platform, Molecular Devices, Sunnyvale, Calif.) and the cell viability in response to various tellurium nanoparticle concentrations was determined. Cell viability was calculated by dividing the average absorbance obtained for each sample by the absorbance of the control sample with no nanoparticles, and then multiplying the result by 100 to obtain percent viability.

1.6. Statistical Analysis

All experiments were done in triplicate (N=3) to ensure reliability and replicability of results. Experimental results were assessed for statistical significance using a students t-test (p≤0.05 being considered significant). All data was presented as mean±standard deviation.

### 2. RESULTS AND DISCUSSION

2.1 Synthesis and Purification of PVP-Coated Tellurium Nanoparticles

A simple, safe method was developed for the synthesis of tellurium nanorods, employing a hydrothermal approach. The presence of the reducing agent, 1-ascorbic acid, in addition to the polymeric material, PVP, within the solvent assisted in the reduction of metallic ions to form nanoparticles. The temperature gradient within the reactor also assisted in the formation of the nanorods [44-45]. Both 1-ascorbic acid and PVP are readily available, environmentally benign, and odorless. Furthermore, the Food and Drug Administration (FDA) has approved PVP for use as a food additive and in pharmaceutical applications. Because these environmentally safe materials were used, no toxic byproducts were produced.

2.2 Characterization of PVP-Coated Tellurium Nanoparticles Via DLS, TEM, and EDS

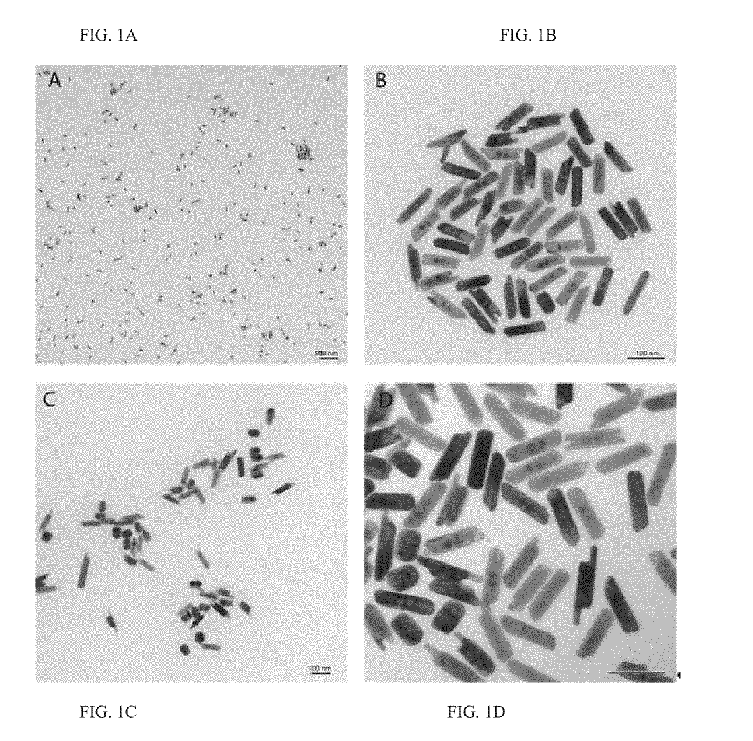

After purification, nanoparticles were characterized using DLS. DLS analysis demonstrated that the nanorods had an effective diameter of 121 nm, with a degree of polydispersity of 0.249. The morphology of the synthesized nanorods was also validated using TEM. A uniform size distribution was observed, with an observed width of 22±3.2 nm and length of 86.3±13.8 nm. Particles were confirmed to be rod-shaped with plain extremes and smooth surfaces. Slight aggregation of the nanoparticles was observed (FIG. 1B), but these aggregates were easily separated with brief sonication (FIG. 1C). From the TEM images, a variation in nanorod thickness was also noted. Distinct nanorod growth phases were also detected as determined by the various lengths of the nanorods, and smaller tellurium particles can be seen encapsulated in PVP. (FIG. 1D).

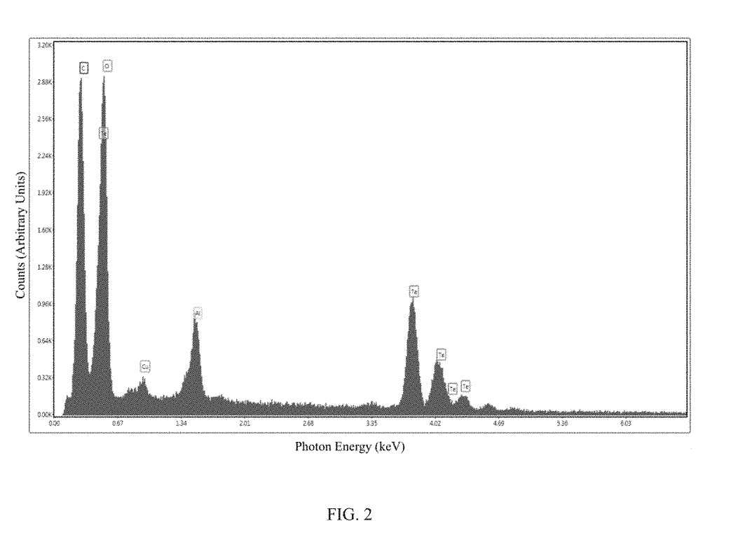

To determine the elemental composition of the nanoparticles, electron dispersive X-ray spectroscopy (EDX) measurements were performed. This characterization confirmed that the electron dense nanoparticles were composed of tellurium as determined by specific tellurium peaks (FIG. 2). Significant oxygen and carbon peaks are also seen, indicating the presence of PVP within the nanorods. The presence of an aluminium peak was also noted and is likely due to the composition of the sample mount employed for measurements. Lastly, a copper peak was also observed as a result of the use of copper grids in sample fixation.

2.3 PVP-Coated Tellurium Nanoparticles Demonstrate Significant Antibacterial Activity Against Both Escherichia coli and Staphylococcus aureus

To determine the antibacterial activity of the PVP-coated tellurium nanoparticles, bacterial growth and colony forming assays were performed using Escherichia coli and Staphylococcus aureus.

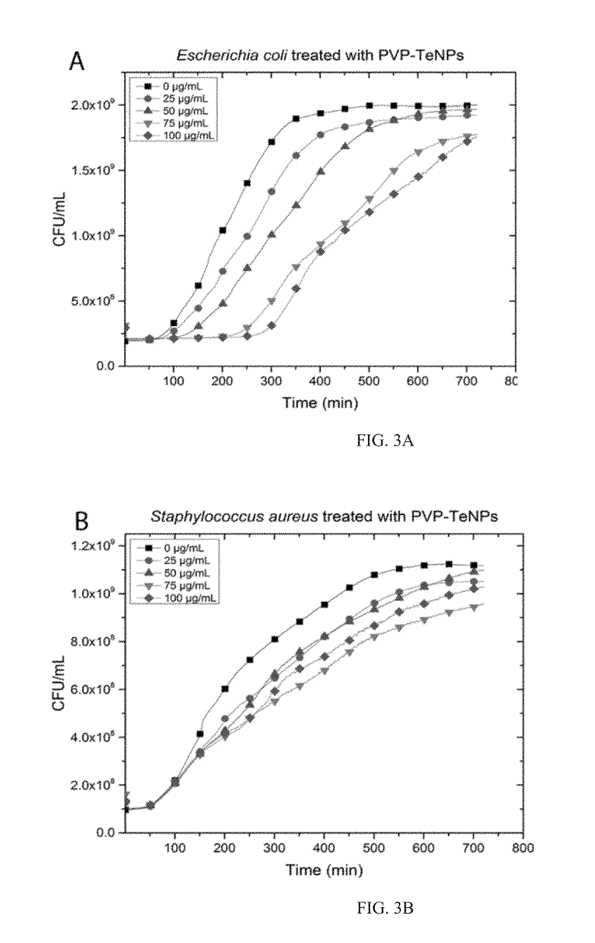

FIGS. 3A and 3B are graphs showing that PVP-coated tellurium nanoparticles in accordance with an embodiment of the invention decrease the growth of Escherichia coli (see FIG. 3A) and Staphylococcus aureus (see FIG. 3B). Growth is shown of a 10⁶ CFU mL⁻¹ suspension of Escherichia coli (FIG. 3A) and Staphylococcus aureus (FIG. 3B) over 24 hours in presence of different concentrations of tellurium nanoparticles. The values represent the mean±standard deviation, N=3.

Nanoparticle concentrations between 25 and 100 μg/mL decayed the growth of Escherichia coli (see FIG. 3A). The level of this decay increased with a rise in nanoparticle concentration.

Similar results were obtained with Staphylococcus aureus (see FIG. 3B), with concentrations between 25 and 100 μg/mL causing a decay in bacterial growth compared to the control. However, this effect was less pronounced than with Escherichia coli.

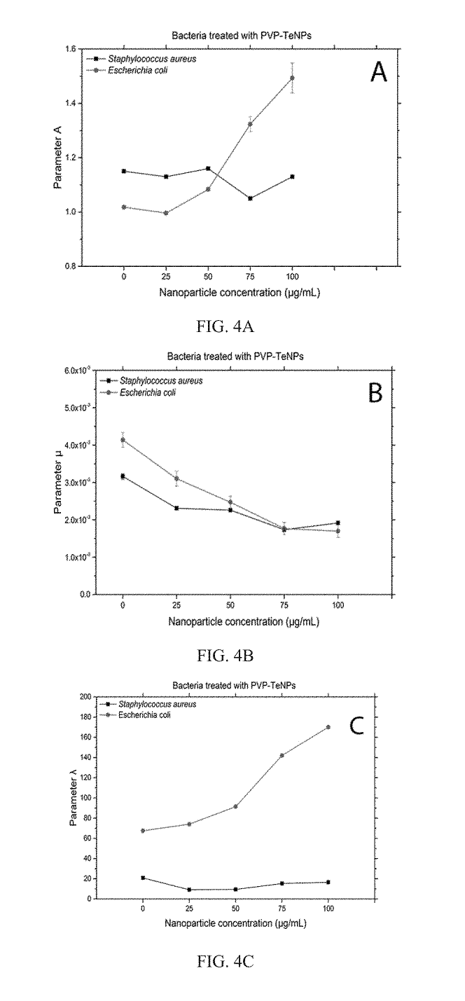

To further investigate the effects of tellurium nanoparticles on bacterial growth, the parameters of the modified Gompertz equation (Eq. 3) were calculated and plotted for analysis (see FIGS. 4A-4C).

FIGS. 4A-4C show graphs of Modified Gompertz equation parameters A, μ, and λ for increasing concentrations of PVP-Tellurium nanoparticles. Growth of a 10⁶ CFU mL⁻¹ suspension of Escherichia coli and Staphylococcus aureus over 24 hours in presence of different concentrations of tellurium nanoparticles are shown. The values represent the mean±standard deviation, N=3.

Parameter A, which represents the maximum bacterial growth, decreased at increasing concentrations. The antibacterial activity was especially noticeable in the case of Staphylococcus aureus. Additionally, larger concentrations of PVP-coated tellurium nanoparticles led to lower maximum bacterial growth in Escherichia coli (FIG. 4A). In general, this decay shows that there is an antibacterial effect of PVP-coated tellurium nanoparticles.

Changes in maximum bacterial growth rate was determined by analyzing the parameter μ (see FIG. 4B). This analysis demonstrated that higher tellurium nanoparticle concentrations resulted in a lower growth rate of the bacteria. This trend was similar in both bacteria.

Lastly, the parameter λ, which represents the lag time in the bacterial growth, or the duration of time where bacteria are adapting themselves to the growth conditions offered by the media, was analyzed (see FIG. 4C). This analysis showed that higher tellurium nanoparticle concentrations led to a larger lag phase in bacterial growth. This was especially visible with Escherichia coli. This suggests that the presence of PVP-coated tellurium nanoparticles delays bacterial maturation, therefore inhibiting bacterial growth.

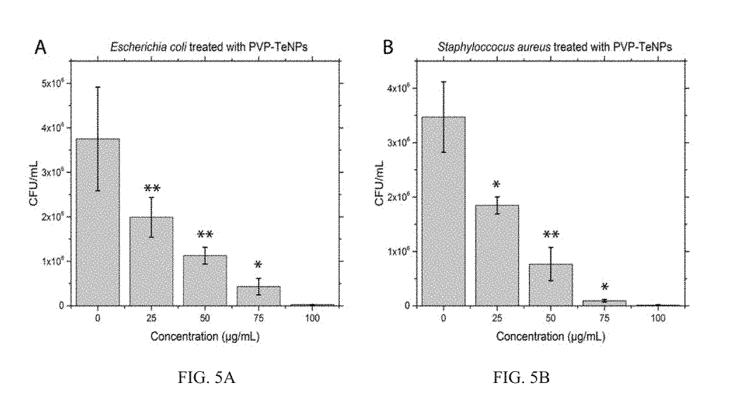

To further assess the antibacterial activity of tellurium nanoparticles, colony forming unit assays were performed. Towards both Escherichia coli and Staphylococcus aureus, increasing concentrations of PVP-coated tellurium nanoparticles induced a significant decay in the number of bacterial colonies. A near total inhibition of bacterial growth was observed at concentrations of 75 and 100 μg/mL (see FIGS. 5A and 5B). Combined with bacterial growth assays, these results demonstrate significant antibacterial properties of tellurium nanoparticles.

FIGS. 5A and 5B are colony counting assays of (FIG. 5A) Escherichia coli and (FIG. 5B) Staphylococcus aureus after being treated for 8 hours with PVP-coated tellurium nanorods. N=3. *p<0.01 versus control (0 μg/mL concentration), **p<0.005 versus control (0 μg/mL concentration).

Without wishing to be bound by any specific theory, the observed antibacterial activity of PVP-coated tellurium nanoparticles may be explained by the increased production of reactive oxygen species (ROS). ROS are chemically reactive agents and free radicals containing oxygen, such as hydroxyl (OH⁻) or superoxide (O₂⁻) groups. Although these species are normally formed as natural byproducts of oxygen metabolism, exposure of cells to metallic nanoparticles can increase ROS concentrations to induce cellular toxicity [46-48]. Therefore, it is hypothesized that the presence of PVP-coated tellurium nanoparticles increased the formation of ROS and, as a consequence, led to a systematic failure of the internal metabolism of the bacteria and subsequent antibacterial activity.

2.4 In Vitro Cytotoxicity of Tellurium Nanoparticles with Human Dermal Fibroblast and Melanoma Cells

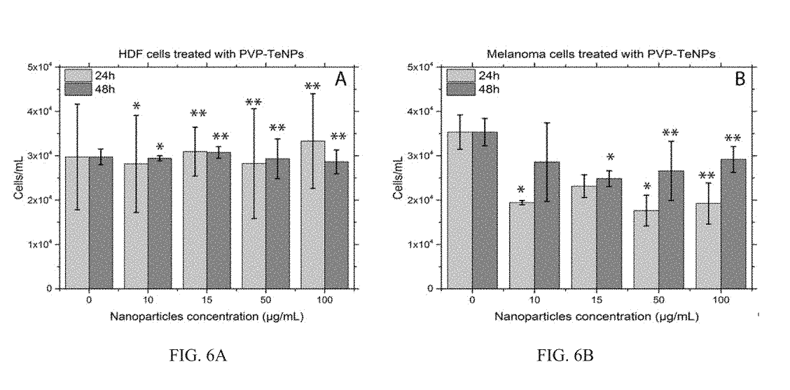

To determine the toxicity of the PVP-coated tellurium nanorods on mammalian cells, in vitro cytotoxicity assays were performed with human dermal fibroblasts (HDF) and human melanoma cells. At nanoparticle concentrations between 10 and 100 μg/mL, an MTS assay demonstrated no significant cytotoxicity in HDF (see FIG. 6A).

FIGS. 6A and 6B are MTS assays on human dermal fibroblast (FIG. 6A) and melanoma (FIG. 6B) cells in the presence of PVP-Tellurium nanoparticles at concentrations ranging from 10-100 μg/mL. N=3. *p<0.01 versus control (0 μg/mL concentration), **p<0.005 versus control (0 μg/mL concentration).

An MTS assay was also carried out with melanoma cells to investigate the potential anticancer activity of the tellurium nanoparticles. Over the same concentration range, there was a significant decay in cell growth within the first 24 hours of incubation, with a cell viability ranging from 55-65% compared to the control (see FIG. 6B). However, after 48 hours of incubation, a rise in cell viability was observed. Cell viabilities ranged between 75-85% compared to the control.

Collectively, these results suggest that PVP-tellurium nanoparticles have substantial antibacterial effects while also having negligible cytotoxic effects on healthy human cells. In addition, as has similarly been seen with other biologically active tellurium compounds such as AS101, the tellurium nanoparticles have inherent anti-cancer properties [26]. A previous study hypothesized that this property is due to the reduction/oxidation potential of tellurium compounds [19]. Collectively such results indicate a promising area for future research, especially considering increased infection rates for patients with cancer due to weakened immune systems.

### 3. CONCLUSION

Many current methods used to synthesize tellurium nanoparticles employ traditional chemistry methods, including the use of toxic chemicals. While effective, these procedures often involve extreme reaction conditions, the production of toxic byproducts, and the need for excessive purification. An alternative approach is to use green nanoparticle synthesis, which utilizes environmentally-safe materials and biologic agents to synthesize nanoparticles without the limitations of chemical approaches. This work demonstrates a green synthesis technique that uses hydrothermal reactions at lower operating temperatures and with environmentally-friendly materials to synthesize PVP-coated tellurium nanorods. The nanorods were characterized using DLS, TEM, and EDS to determine their size, morphology, and composition. The resulting nanoparticles showed a relatively homogeneous size distribution, with an observed width of 22±3.2 nm and an observed length of 86.3±13.8 nm. Antibacterial assays demonstrated significant antibacterial activity towards both gram negative and gram positive bacteria for a range of nanoparticle concentrations between 25 and 100 μg/mL. Additionally, the cytotoxicity of the nanoparticles was tested against human dermal fibroblasts. No significant cytotoxicity was found at all nanoparticle concentrations. Lastly, it was found that the tellurium nanoparticles possessed anticancer properties towards melanoma cells, showing a decay in the cell viability after 24 hours of nanoparticle incubation.

In summary, PVP-coated tellurium nanorods in accordance with an embodiment of the invention, produced via a hydrothermal, green synthesis method represent a promising nanomaterial due to their antibacterial and anticancer activity.

### REFERENCES

[1] Ventola C L. The Antibiotic Resistance Crisis: Part 1: Causes and Threats. Pharmacy and Therapeutics. 2015; 40(4):277-283.
[2] Lushniak, Boris D. “Antibiotic Resistance: A Public Health Crisis.” Public Health Reports, vol. 129, no. 4, 2014, pp. 314-316, doi:10.1177/003335491412900402.
[3] Huttner, Benedikt, and Stephan Harbarth. Healthcare-Associated Infections and Emergence of Antibiotic Resistance. Oxford Medicine Online, 2017, doi:10.1093/med/9780198701590.003.0086.
[4] Davey, P., Marwick, C. A., Scott, C. L., Charani, E., Mcneil, K., Brown, E., Gould, I. M., Ramsay, C. R., Michie, S. “Interventions to Improve Antibiotic Prescribing Practices for Hospital Inpatients.” Cochrane Database of Systematic Reviews, 2013, doi:10.1002/14651858.cd003543.pub3.
[5] Centers for Disease Control and Prevention. 2014 National and State Healthcare-Associated Infections Progress Report. Published March, 2016. Available at www.cdc.gov/hai/progress-report/index.html.
[6] Rai, M. K., Deshmukh, S. D., Ingle, A. P., Gade, A. K. “Silver Nanoparticles: the Powerful Nanoweapon against Multidrug-Resistant Bacteria.” Journal of Applied Microbiology, vol. 112, no. 5, 2012, pp. 841-852., doi:10.1111/j.1365-2672.2012.05253.x.
[7] Lara, H., Ayala-Nunez, N., Ixtepan Turrent, L., Rodriguez Padilla, C. “Bactericidal Effect of Silver Nanoparticles against Multidrug-Resistant Bacteria.” World Journal of Microbiology and Biotechnology, vol. 26, no. 4, 2009, pp. 615-621., doi:10.1007/s11274-009-0211-3.
[8] Li, X., Robinson, S., Gupta, A., Saha, K., Jiang, Z., Moyano, D., Sahar, A., Riley, M., Rotello, V. “Functional Gold Nanoparticles as Potent Antimicrobial Agents against Multi-Drug-Resistant Bacteria.” ACS Nano, vol. 8, no. 10, 2014, pp. 10682-10686., doi:10.1021/nn5042625.
[9] Cao, Huiliang. Silver Nanoparticles for Antibacterial Devices: Biocompatibility and Toxicity. CRC Press, 2017.
[10] Yu, Q., Li, J., Zhang, Y., Wang, Y., Liu, L., Li, M. Inhibition of gold nanoparticles (AuNPs) on pathogenic biofilm formation and invasion to host cells. Sci Rep. 2016; 6:26667.
[11] Li, M., Zhu, L., Lin, D. Toxicity of ZnO nanoparticles to Escherichia coli: mechanism and the influence of medium components. Environmental Science Tech. 2011; 45(5):1977-1983.
[12] Parveen, K., Banse, V., Ledwani, L. (2016). Green synthesis of nanoparticles: Their advantages and disadvantages. AIP Conference Proceedings, 1724(1), 20048. https://doi.org/10.1063/1.4945168
[13] Virkutyte, Jurate, and Rajender S. Varma. “Environmentally Friendly Preparation of Metal Nanoparticles.” Green Chemistry Series: Sustainable Preparation of Metal Nanoparticles, pp. 7-33., doi:10.1039/9781849735469-00007.
[14] Jenkins, Scott. “Green Chemistry Principles.” Process Intensification for Green Chemistry: Engineering Solutions for Sustainable Chemical Processing, edited by Kamelia V. K. Boodhoo and Adam Harvey, Wiley, 2013, pp. 33-58.
[15] Medina Cruz D., Mi G., Webster T. J. “Synthesis and Characterization of Biogenic Selenium Nanoparticles with Antimicrobial Properties Made by Staphylococcus aureus, Methicillin-resistant Staphylococcus aureus (MRSA), Escherichia coli and Pseudomonas aeruginosa.” J Biomed Mater Res A (2018).
[16] Fathima, B. Sogra, and Raj Mohan Balakrishnan. “Biosynthesis and Optimization of Silver Nanoparticles by Endophytic Fungus Fusarium Solani.” Materials Letters, vol. 132, 2014, pp. 428-431., doi:10.1016/j.matlet.2014.06.143.
[17] Ezonaro, E., Elampis, S., Turner, R. J., S. Quazi, S. J., Evallini, G. “Biogenic Selenium and Tellurium Nanoparticles Synthesized by Environmental Microbial Isolates Efficaciously Inhibit Bacterial Planktonic Cultures and Biofilms.” Frontiers in Microbiology, vol. 6, 2015, doi:10.3389/fmicb.2015.00584
[18] Virkutyte, J., and Rajender, S. V. “Green Synthesis of Metal Nanoparticles: Biodegradable Polymers and Enzymes in Stabilization and Surface Functionalization.” Chemical Science, vol. 2, no. 5, 2011, pp. 837-846., doi:10.1039/cOsc00338g.
[19] Ba, L. A., Döring, M., Jamier, V., and Jacob, C. (2010). Tellurium: an element with great biological potency and potential. Organic and Biomolecular Chemistry, 8(19), 4203. https://doi.org/10.1039/c0ob00086h
[20] Mousavi-Kamazani, M., Rahmatolahzadeh, R., Amin Shobeiri, S., and Beshkar, F. (2017). Sonochemical synthesis, formation mechanism, and solar cell application of tellurium nanoparticles. Ultrasonics Sonochemistry, 39(March), 233-239. https://doi.org/10.1016/j.ultsonch.2017.04.031
[21] Ogra, Y. (2017). Biology and toxicology of tellurium explored by speciation analysis. Metallomics, 9, 435-441. https://doi.org/10.1039/C7MT00022G
[22] Cunha, R. L. O. R., Gouvea, I. E., and Juliano, L. (2009). A glimpse on biological activities of tellurium compounds. Anais Da Academia Brasileira de Ciencias, 81(3), 393-407. https://doi.org/10.1590/S0001-37652009000300006
[23] Chasteen Fuentes, D. E., Tantalean, J. C., and Vasquez, C. C., T. G. (2009). Tellurite: history, oxidative stress, and molecular mechanisms of resistance. FEMS Microbiol Rev, 33, 820-832. https://doi.org/10.1111/j.1574-6976.2009.00177.x
[24] Sredni, B. (2012). Immunomodulating tellurium compounds as anti-cancer agents. Seminars in Cancer Biology, 22(1), 60-69. https://doi.org/10.1016/j.semcancer.2011.12.003
[25] Zare, B., Nami, M., and Shahverdi, A. R. (2017). Tracing Tellurium and Its Nanostructures in Biology. Biological Trace Element Research, (March), 1-11. https://doi.org/10.1007/s12011-017-1006-2
[26] Seng, Hoi-Ling, and Edward R. T. Tiekink. “Anti-Cancer Potential of Selenium- and Tellurium-Containing Species: Opportunities Abound!” Applied Organometallic Chemistry, vol. 26, no. 12, 2012, pp. 655-662., doi:10.1002/aoc.2928.
[27] Ikemoto, H., Goyo, A., and Miyanaga, T. (2011). Size Dependence of the Local Structure and Atomic Correlations in Tellurium Nanoparticles. The Journal of Physical Chemistry C, 115(7), 2931-2937. https://doi.org/10.1021/jp107475x
[28] Yuan, Q. L., Yin, H. Y., and Nie, Q. L. (2013). Nanostructured tellurium semiconductor: from nanoparticles to nanorods. J Exp Nanosci, 8(December), 931-936. https://doi.org/10.1080/17458080.2011.620021
[29] Borghese, R., Brucale, M., Fortunato, G., Lanzi, M., Mezzi, A., Valle, F.,
Zannoni, D. (2016). Extracellular production of tellurium nanoparticles by the photosynthetic bacterium Rhodobacter capsulatus. Journal of Hazardous Materials, 309, 202-209. https://doi.org/10.1016/j.jhazmat.2016.02.011
[30] Forootanfar, H., Amirpour-Rostami, S., Jafari, M., Forootanfar, A., Yousefizadeh, Z., and Shakibaie, M. (2015). Microbial-assisted synthesis and evaluation the cytotoxic effect of tellurium nanorods. Materials Science and Engineering C, 49, 183-189. https://doi.org/10.1016/j.msec.2014.12.078
[31] Pugin, B., Cornejo, F. A., Munoz-Diaz, P., Munoz-Villagrán, C. M., Vargas-Perez, J. I., Arenas, F. A., and Vasquez, C. C. (2014). Glutathione reductase-mediated synthesis of tellurium-containing nanostructures exhibiting antibacterial properties. Applied and Environmental Microbiology, 80(22), 7061-7070. https://doi.org/10.1128/AEM.02207-14
[32] Ramos-Ruiz, A., Field, J. A., Wilkening, J. V., and Sierra-Alvarez, R. (2016). Recovery of Elemental Tellurium Nanoparticles by the Reduction of Tellurium Oxyanions in a Methanogenic Microbial Consortium. Environmental Science and Technology, 50(3), 1492-1500. https://doi.org/10.1021/acs.est.5b04074
[33] Zare, B., Faramarzi, M. A., Sepehrizadeh, Z., Shakibaie, M., Rezaie, S., and Shahverdi, A. R. (2012). Biosynthesis and recovery of rod-shaped tellurium nanoparticles and their bactericidal activities. Materials Research Bulletin, 47(11), 3719-3725. https://doi.org/10.1016/j.materresbull.0.2012.06.034
[34] Wang, J., Fang, M., Fei, G. T., Liu, M., Shang, G. L., and De Zhang, L. (2016). Te hexagonal nanotubes: formation and optical properties. Journal of Materials Science, 51(15), 7170-7178. https://doi.org/10.1007/s10853-016-9997-1
[35] He, W., Krejci, A., Lin, J., Osmulski, M. E., and Dickerson, J. H. (2011). A facile synthesis of Te nanoparticles with binary size distribution by green chemistry. Nanoscale, 3(4), 1523-1525. https://doi.org/10.1039/c1nr10025d
[36] Zheng, R., Cheng, W., Wang, E., and Dong, S. (2004). Synthesis of tellurium nanorods via spontaneous oxidation of NaHTe at room temperature. Chemical Physics Letters, 395(4-6), 302-305. https://doi.org/10.1016/j.cplett.2004.07.091
[37] Taylor, D. E. (1999). Bacterial tellurite resistance. Trends in Microbiology, 7(3), 111-115. https://doi.org/10.1016/50966-842X(99)01454-7
[38] Walter, E. G., and Taylor, D. E. “Plasmid-Mediated Resistance to Tellurite: Expressed and Cryptic.” Plasmid, vol. 27, no. 1, 1992, pp. 52-64., doi:10.1016/0147-619x(92)90006-v.
[39] Zhang, Y., Peng, H., Huang, W., Zhou, Y. Zhang, X., Yan, D. “Hyperbranched Poly(Amidoamine) as the Stabilizer and Reductant to Prepare Colloid Silver Nanoparticles in Situ and Their Antibacterial Activity.” The Journal of Physical Chemistry C, vol. 112, no. 7, 2008, pp. 2330-2336., doi:10.1021/jp075436g.
[40] Liu, L., Yang, J., Xie, J., Luo, Z., Jiang, J. “The Potent Antimicrobial Properties of Cell Penetrating Peptide-Conjugated Silver Nanoparticles with Excellent Selectivity for Gram-Positive Bacteria over Erythrocytes.” Nanoscale, vol. 5, no. 9, 2013, p. 3834., doi:10.1039/c3nr34254a.
[41] Gacem, N., and Diao, P. (2013). Effect of solvent polarity on the assembly behavior of PVP coated rhodium nanoparticles. Colloids and Surfaces A: Physicochemical and Engineering Aspects, 417, 32-38. https://doi.org/10.1016/j.colsurfa.2012.10.055
[42] Jianfeng, Y., Guisheng, Z., Anming, H., and Norman, Z. Y. (2011). Preparation of PVP coated Cu NPs and the application for low-temperature bonding. Journal of Materials Chemistry, 21(1), 15981-15986. https://doi.org/10.1039/cljm12108a
[43] Zannoni, D., Borsetti, F., Harrison, J. J., and Turner, R. J. (2007). The Bacterial Response to the Chalcogen Metalloids Se and Te. Advances in Microbial Physiology (Vol. 53). Doi:10.1016/S0065-2911(07)53001-8
[44] Polsongkram, D., Chamninok, P., Pukird, S., Chow, L., Lupan, O., Chai, G., Khallaf, H., Park, S., Schulte, A. “Effect of Synthesis Conditions on the Growth of ZnO Nanorods via Hydrothermal Method.” Physica B: Condensed Matter, vol. 403, no. 19-20, 2008, pp. 3713-3717., doi:10.1016/j.physb.2008.06.020.
[45] Liu, Z., Hu, Z., Liang, J., Yang, Y., Peng, S., Qian, Y. “Size-Controlled Synthesis and Growth Mechanism of Monodisperse Tellurium Nanorods by a Surfactant-Assisted Method.” Langmuir, vol. 20, no. 1, 2004, pp. 214-218., doi:10.1021/1a035160d.
[46] Suresh, Anil K. “Co-Relating Metallic Nanoparticle Characteristics and Bacterial Toxicity.” Springer Briefs in Molecular Science, 2015, doi:10.1007/978-3-319-16796-1.
[47] Guo, D., Zhu, L., Huang, Z., Zhou, H., Ge, Y., Ma, W. Gu, N. (2013). Anti-leukemia activity of PVP-coated silver nanoparticles via generation of reactive oxygen species and release of silver ions. Biomaterials, 34(32), 7884-7894. https://doi.org/10.1016/j.biomaterials.2013.07.015
[48] Dayem, A. A., Hossain, M. K., Lee, S. B., Kim, K., Saha, S. K., Yang, G. M., Choi, H. Y., Cho, S. G. “The Role of Reactive Oxygen Species (ROS) in the Biological Activities of Metallic Nanoparticles.” International Journal of Molecular Sciences, vol. 18, no. 1, October 2017, p. 120., doi:10.3390/ijms18010120.1

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A nanoparticle, the nanoparticle comprising:
tellurium; and
polyvinylpyrrolidone (PVP);
the nanoparticle having a rod shape.

2. The nanoparticle of claim 1, wherein the nanoparticle has a length of between about 72 nm and about 101 nm, and a width of between about 18 nm and about 26 nm.

3. The nanoparticle of claim 1, wherein the nanoparticle has a length and a width such that an aspect ratio of the length of the nanoparticle to the width of the nanoparticle is between about 2.8 and about 5.3.

4. The nanoparticle of claim 1, wherein the nanoparticle has a zeta potential of between about −25 mV and about −35 mV.

5. The nanoparticle of claim 1, wherein the nanoparticle comprises a surface that is functionalized by the polyvinylpyrrolidone (PVP).

6. The nanoparticle of claim 5, wherein the surface of the nanoparticle is coated with the polyvinylpyrrolidone (PVP).

7.-11. (canceled)

12. A composition, the composition comprising:
a carrier medium; and
a plurality of nanoparticles comprising: (i) tellurium, and (ii) polyvinylpyrrolidone (PVP), the nanoparticles comprising a rod shape;
wherein the plurality of nanoparticles is present in the carrier medium in a concentration of between about 10 micrograms per milliliter (μg/mL) and about 100 micrograms per milliliter (μg/mL).

13. The composition of claim 12, wherein the plurality of nanoparticles is present in a concentration of between about 25 micrograms per milliliter (μg/mL) and about 100 micrograms per milliliter (μg/mL) in the carrier medium.

14. The composition of claim 12, wherein the carrier medium comprises a solvent.

15. The composition of claim 14, wherein the solvent comprises an aqueous solution.

16. The composition of claim 12, comprising a pharmaceutical composition, and wherein the carrier medium comprises a pharmaceutically acceptable excipient.

17. (canceled)

18. The composition of claim 12, wherein the nanoparticles have a length of between about 72 nm and about 101 nm, and a width of between about 18 nm and about 26 nm.

19. The composition of claim 12, wherein the nanoparticles have a length and a width such that an aspect ratio of the length of each nanoparticle to its width is between about 2.8 and about 5.3.

20. The composition of claim 12, wherein the nanoparticles have a zeta potential of between about −25 mV and about −35 mV.

21.-27. (canceled)

28. A device comprising an antibacterial or anticancer surface, the device comprising:
a coating on at least part of a surface of the device, the coating comprising a plurality of nanoparticles comprising: (i) tellurium, and (ii) polyvinylpyrrolidone (PVP), the nanoparticles comprising a rod shape.

29. The device of claim 28, wherein the device comprises medical equipment.

30. The device of claim 29, wherein the medical equipment comprises at least one of: a medical implant, a surgical instrument, a medical tubing, and a component of a medical operating theater.

31. The device of claim 28, wherein the nanoparticles have a length of between about 72 nm and about 101 nm, and a width of between about 18 nm and about 26 nm.

32. The device of claim 28, wherein the nanoparticles have a length and a width such that an aspect ratio of the length of the nanoparticle to the width of the nanoparticle is between about 2.8 and about 5.3.

33. The device of claim 28, wherein the nanoparticles have a zeta potential of between about −25 mV and about −35 mV.

34.-35. (canceled)

36. A process of preparing a nanoparticle, the process comprising:
performing a hydrothermal reduction reaction of a tellurium-containing compound with polyvinylpyrrolidone (PVP) and an aqueous solution of an organic compound, thereby producing a solution of nanoparticles comprising: (i) tellurium, and (ii) polyvinylpyrrolidone (PVP), the nanoparticles having a rod shape; and
performing a physical purification of the nanoparticles from the solution.

37. The process of claim 36, wherein the tellurium-containing compound comprises a salt of tellurium.

38. (canceled)

39. The process of claim 36, wherein the organic compound comprises an organic acid.

40. (canceled)

41. The process of claim 36, wherein the physical purification comprises at least one of: a drying of the solution, a washing, and a lyophilization.

42.-47. (canceled)

48. A method of treating cancer in a patient in need thereof, comprising:
administering an effective amount of a pharmaceutical composition comprising a nanoparticle, the pharmaceutical composition comprising: (i) a carrier medium; (ii) a plurality of nanoparticles comprising tellurium, and polyvinylpyrrolidone (PVP), the nanoparticles comprising a rod shape; and (iii) a pharmaceutically acceptable excipient;
wherein the plurality of nanoparticles is present in the carrier medium in a concentration of between about 10 micrograms per milliliter (μg/mL) and about 100 micrograms per milliliter (μg/mL).

49.-57. (canceled)

58. The method of claim 48, wherein the nanoparticles have cytotoxic effects against human melanoma cells.

59. A method of treating a bacterial infection in a patient in need thereof, comprising:
administering an effective amount of a pharmaceutical composition comprising a nanoparticle, the pharmaceutical composition comprising: (i) a carrier medium; (ii) a plurality of nanoparticles comprising tellurium, and polyvinylpyrrolidone (PVP), the nanoparticles comprising a rod shape; and (iii) a pharmaceutically acceptable excipient;
wherein the plurality of nanoparticles is present in the carrier medium in a concentration of between about 25 micrograms per milliliter (μg/mL) and about 100 micrograms per milliliter (μg/mL).

60.-68. (canceled)

69. The method of claim 59, wherein the nanoparticles have antibacterial properties against at least one of Gram-positive and Gram-negative bacteria.

70. The method of claim 59, wherein the nanoparticles have antibacterial properties against antibiotic resistant bacteria.

71. The method of claim 59, wherein the nanoparticles have antibacterial properties against at least one of: Escherichia coli, Staphylococcus aureus, methicillin-resistant Staphylococcus aureus, and multidrug-resistant Escherichia coli.
